(19) United States
(12) Patent Application Publication
(10) Pub. No.: US 2006/0052263 A1
(43) Pub. Date: Mar. 9, 2006

(54) Soap preparation with air bubbles

(75) Inventors: Michael Roreger, NEUWIED (DE); Iris Schnitzler, Bonn (DE); Qian-Yi Li, Bonn (DE)

Correspondence Address: Frommer Lawrence & Haug, 745 Fifth Avenue, New York, NY, 10151, US

(73) Assignee: SCS SKIN CARE SYSTEMS GMBH

(21) Appl. No.: 10/526,570

(22) PCT Filed: Sep. 2, 2003
(86) PCT No.: PCT/EP03/09712

§ 371 Date: Mar. 4, 2005

(30) Foreign Application Priority Data
Sep. 7, 2002 (DE) 102 41 597.8

(51) Int. Cl. A61K 8/02

(52) U.S. Cl. 510/141; 510/152

(57) ABSTRACT

Disclosed is a solid soap preparation which can dissolve very quickly when it comes into contact with water and which enables exact dosing of a specific amount of soap. The soap preparation contains air bubbles and thus exhibits a certain degree of elasticity and plasticity.

The present invention relates to a solid soap preparation which can preferably be configured in the form of a sheet. The soap preparation comprises air bubbles, but can also comprise fragrances and/or other cosmetic active ingredients.

The use of soap serves the purpose of releasing soilings from surfaces due to the wetting ability of aqueous soap solutions and, in so doing, of removing them. For the body care sector, soap is supplied in solid form (as soap bar, curd soap, soap flakes) and in liquid and/or flowable form (solution, shampoo, gel).

CN 1,134,450 A discloses a rapidly-dissolving soap in the form of a film and its production method. The soap consists of a water-soluble polymer (as film former) negatively charged and nonionic surfactants, complex salts of iodine as disinfectants, skin-nourishing agents and fillers. The soap in the form of a film is obtained by mixing the materials, dissolving them in water, converting them into film form, drying and cutting. The presence of air bubbles in this soap preparation is not disclosed.

A disadvantage of the products which are supplied in solid form as soap bar is the slow dissolution in water, which is also a hindrance to the formation of micelles and other association colloids.

A disadvantage of the products which are supplied in liquid and/or flowable form is the difficult dosability of the soap for the user. On account of the liquid nature, overdoses may result which, if used in the body care sector, may be associated with disadvantageous effects due to local skin irritations.

A further disadvantage of the liquid and/or flowable soap preparations is that the high solvent content (water, alcohol, glycerol etc.) has a disadvantageous effect on the manufacturing and transportation costs.

It is an object of the present invention to provide a product through which soap is made available in a predosed amount. The product should dissolve rapidly, be simple and cost-effective to produce and offer high application convenience. In the body care sector, the product is preferably suitable for a single use.

The object is achieved by a soap preparation which is solid and preferably elastic and plastic. The soap preparation comprises soap and at least one film- or backbone-forming polymer. In addition, the soap preparation comprises air bubbles which are preferably introduced as a result of the production method. The soap preparation thus forms a solid foam.

One feature of the soap preparation is that it is solid which, for the purposes of the present description, is understood as meaning only that it does not have a liquid or flowable consistency. Due to the presence of the air bubbles on a molecular level, the soap preparation is a dispersion, i.e. a substance mixture consisting of two phases in which the air bubbles form the disperse gaseous phase which are distributed in very fine form in the mixture of soap and film- or backbone-forming polymer (dispersant).

The soap preparation may preferably be elastic and plastic. Elasticity and plasticity are used by the person skilled in the art to refer to the property of solid bodies to change their external shape due to an externally acting effect of force (tensile stress, compression). While the property “elastic” means that the body reassumes its former shape after this external force effect has been lifted (=reversible deformation), the property “plastic” means that the body retains a changed external shape as a result of the external effect of force (=irreversible deformation). Examples of elastic materials are vulcanized rubber, natural rubber etc.; examples of plastic materials are clay, metals etc.

The soap preparation has then the property of being both elastic and plastic. This behavior depends essentially on the force which is exerted onto the soap preparation, the external shape of the soap preparation, and also on the size and the number of the air bubbles present in the soap preparation. This ability can also be controlled via the properties of the film- or backbone-forming polymer (cohesion, molecular weight, degree of crosslinking etc.). The presence of plasticizers, fats, oils, surfactants etc. can likewise influence the elasticity and/or plasticity of the soap preparation. Due to the plasticity and the elasticity, the soap preparation, particularly in a sheetlike or striplike configuration, has considerably marked flexibility.

Preferably, the soap preparation is designed such that, when a slight external force (which corresponds, for example, to the pressure to be achieved when gently pressing together thumb and forefinger) is exerted, it returns again completely to the original shape as is known from a sponge. However, if greater pressures are applied, the solid soap preparation can be compressed such that a large part of the air bubbles present there are also pressed out. This corresponds to the case of typical plasticity since such a deformation is irreversible.

The soap preparation can have an expandability of up to 25%, but the expandability is preferably between 2 and 10%.

A further property of the soap preparation is its density, which is below 1. The density is influenced virtually exclusively by the number and the size of the air bubbles. The size and the number of the air bubbles in the soap preparation may essentially be controlled by the production method. Preferably, the air bubbles have a size of less than about 100 μm, particularly preferably less than 30 μm. However, it should be taken into consideration that even significantly smaller air bubbles (in the nanometer range) may be present in the soap preparation. As a result of these qualities, the density of the soap preparation can thus be between 1 and 0.05 g/cm³, preferably between 0.7 and 0.1 g/cm³.

It may be noted that the air bubbles preferably consist of natural ambient air. However, they can also comprise other gaseous constituents or be constructed therefrom, such as, for example, nitrogen (N₂), carbon dioxide (CO₂), a protective gas such as helium (He), a propellant gas such as, for example, butane, pentane, dichlorodifluoromethane etc., and mixtures thereof.

For the purposes of the present description, a soap is understood as meaning a water-soluble sodium, potassium or ammonium salt of the saturated or unsaturated higher fatty acids, the resin acids of colophony, the naphthenic acids, and mixtures thereof. In particular, these include sodium stearate, sodium palmitate and sodium oleate. A soap can thus be regarded as being any agent which is able to dissolve in water, to reduce the surface tension of the water, to form micelles in aqueous solution, and in the form of an aqueous solution to bring about wetting of soil particles and a reduction in the adhesion of these soil particles. The (ionic and nonionic) surfactants known to the person skilled in the art are also types of such soaps.

The soap preparation can also comprise the additives for fine soaps known to the person skilled in the art.

Of suitability here are refatting agents, skin protectants, skin care agents, fragrances, essential oils, foam boosters, glycerol, polyols, matting agents (such as TiO₂), stabilizers, antioxidants, fragrances (preferably water-soluble), dyes, antimicrobial additives, exfoliants (kaolin, Neuburg silica chalk, kieselguhr, polyethylene particles etc.) and disinfectants. In a particular embodiment, however, they may be free from preservatives and/or antimicrobial additives, which may mean a further advantage over the soap preparations present in liquid and/or flowable form.

Upon contact with water, the soap preparation can dissolve very rapidly due to the air bubbles present therein. A sheetlike or striplike nature of the soap preparation can intensify this dissolution behavior in the sense of a desired, even more rapid dissolution. The dissolution rate of the soap preparation is in the range from a few seconds to several, i.e. 2 to 5, minutes. Mechanical stress (e.g. rubbing with the hands) can increase the rate of the dissolution process. A few seconds, i.e. very rapid dissolution, is understood as meaning that the soap preparation dissolves completely in water in less than 15 seconds, preferably in less than 10 seconds and particularly preferably in less than 5 seconds. This period of time which is required to dissolve the soap preparation in an aqueous medium is termed the dissolution time. It is dependent inter alia on the number and the size of the air bubbles present in the soap preparation and can, correspondingly, be controlled via the production method for a desired dissolution time.

The soap preparation can additionally also comprise at least one cosmetic active ingredient, such as, for example, a skin oil, a skin care agent and/or a skin protectant. A suitable cosmetic active ingredient may also be allantoin, aloe vera, panthenol, provitamin 5, vitamin E and mixtures thereof.

The skin oils include Cevenyl, calendula oil CLR, Cetiol, Cosmetic Liquid, Cosmetic natural oil, Cosmetol, Crodamol, Fluilan, cyclal, di-2-hexyl tartrate, diisopropylidene triglycerol monostearate, 11,14-dioctyltetracosane, ethyl oleate, fractionated coconut oil BP, rosehip seed oil, isodecane, isodecanoic esters, isohexaoctacontane, Isopar, javanicus oil, jojoba oil, Joleo, cherry stone oil, Kristole, kukui nut oil, linoleic ethyl ester, Liquid Base, Liquilan, Luvitol EHO, Mazula, Miglyol, Myritol 318, mink amido-propyldimethylamine lactate, mink oil fatty acid ethyl ester, mink oil polyethylene glycol ester, nonanol, 2-ethylhexyl nonanoate, octyl neopentanoate, octyl octanoate, octyl pelargonate, olive oil fatty acid ester, Panalane L-14A, Patlac IL, Plant oil CLR, polyethylene glycol(7) glyceryl cocoate, polyisoprene, Prisorine, Porbutyl, rice oils, Reisogran, silicone oils, sperm oil (substitute products), super refined olive oil, Tegosoft oils and triisononanoin.

The skin care agents include Abil WE-09, Alcolose W 2, allantoin, Arosulf CL-A1, Bibranol, Biocorno, bisdiglyceryl ether, cholesterol ester, cholesterol polyglycol ether, cholesterol-siloxane compounds, cholesteryl oleate, Choleth, Chrestalan, Clearcol, coconut fatty acid 2-ethylhexyl ester, Collapuron DAK, Condipon, decaglycerol monooleate monosuccinate, dextran fatty acid ester, diacetin, dicyclohexyalkanes, 1,5-dimethyl-2-isopentylhexanol fatty acid ester, dioctyl maleate, Dow Corning 225C, egg oil, Epadermasterole, Epigran, Epikuron, Estalan, ethyl avocadate, fatty acid dextrin ester, fatty acid diester, Fitoderm, Fluid E-370, Fomblin, Gafquat, Gluadin, glyceryl 3,5,6-trimethylhexanolate, guadine, urea-D-glucuronic acid condensate, cis-6-hexadecenoic acid, hexaglycerol distearate tetraacetate, hexa-glycerol hexastearate diacetate, 2,6,10,15,19,23-hexa-methyltetracosane, Hexamol G-810, bis(2-hexyldecyl tartrate), Hydagen P, Hydrocell YP-30, Hydrotriticum QM, hydroxyethylcellulose, Isodragol, isostearic acid lauryl ester, jojoba butter, Jordaquat JO-50, cocoa fruit juice, carrot oil, Katsernol, Kemester, levulinic acid, Lanacid, Lanesta, Lanoil, Lanolina C 500, Lantrol 1673, lecithin products, Lipocutin, LipoHyParts, liposamic acids, Liposols, Lipotrofina A, Luteofilla, menhaden oil, Mesil, methylheptadecanoic acid, Monaquat, myristic 2-octyldodecyl ester, Naetex Q, Natipide II, sodium lactate methylsilanol, sodium lauryl glutamate, sodium stearyl 2-lactylate, Necon DLD, Nerzolane, octadecanoic 9-octadecenyl ester, octadecyl vinyl ether, oleyl 2-hydroxypropionate, oleylpalmitylpalmitolamidopropyl derivatives, Phosal, phospholipid EFA, Phospholipon, polyamino sugar condensate, polybutene, polydecenes, Polymer 28-4979, polymethacrylamidopropyltrimethylammonium chloride, polyquaternium-n, polyvinylpyrrolidone, Prolaurin, L-pyroglutamic acid, Quatrisoft LM-200, Sebopessina, Secol, silk amino acids, silk fibroin, sericin, silicone fatty acid ester, siloxane copolymers, soya stearols, sorbitol sulfate, super sterol ester, stearic acid dimethylammonium chloride, Stearone, Surfactol Q series, tetrabutoxypropylmethicone, peat wax, Trifat S-308, Turtle Oil R-Trixene, Usnagran, Visonoil-R and Wickenol 535 Vita Cos.

The skin protectants include Abil Wax 9809, N-acylamino acid salts, Ajicoat SPQ, aluminum hydroxide, caseine, Ceresperse water dispersible waxes, Dermol, Dermolan L neutral, Eucornol, Finebase, skin protectant O-48-G, Lauridit, linoleic acid (dimerized), perfluoropolyether, polyvinyl alcohol, polyvinylpyrrolidone triacontene polymer, Praestabitol V, Quick Break, Revitalin, Rewoderm S 1330, Sebosan S, starch ester, stearyl heptanoate and styrene-maleic acid copolymer.

The soap preparation has a high use comfort when it is sheetlike or striplike. Sheetlike (or striplike) embodiments are to be regarded as being those in which the ratio of thickness to width (or length, respectively) is in each case in the range above 1:100, preferably above 1:1000. The thickness of a preferred embodiment should not exceed 5 mm. Particularly preferred embodiments have a thickness of 100 μm to 2 mm and widths (or lengths) between 1 and 10 cm.

In a particular embodiment, the soap preparation comprises a foam former. This is understood as meaning a substance (or a mixture of substances) which is able to improve the formation of air bubbles in aqueous solutions when using mechanical methods (stirring, bubbling etc.) and to prolong the existence of these air bubbles in the aqueous mixture and thus to stabilize them. The presence of the film- or backbone-forming polymer then ensures that, following removal of the water content, these air bubbles are incorporated permanently into the soap composition, the foam formed preferably being a closed-cell foam. Such foam formers are known to the person skilled in the art, in particular suitable surfactants or other interface-active substances are suitable for this purpose. The surfactants (washing-active substances) are divided into anionic surfactants (e.g. soap, linear alkyl-benzenesulfonates), cationic surfactants, nonionic surfactants and amphoteric surfactants depending on their chemical structure. Of particular suitability are linear alkylsulfonates (LAS) with an alkyl length of 10-13 carbon atoms. LAS (C10-13) is thus a mixture of substances which is composed of 4 alkyl chain homologs (C10, C11, C12 and C13). The particular LAS homologs are characterized by different properties. One of the best known surfactants is, for example, sodium laureth sulfate.

The soap preparation comprises at least one film- or backbone-forming polymer. Of suitability here are certain natural and synthetic polymers, in particular polyamides, polyacrylates, polyamino acids, polyvinyl acetate, polyvinyl alcohol, polyethylene glycols, polyvinylpyrrolidones, pullulan, alginic acid, starch, cellulose and cellulose derivatives. The polymer which has film-forming and/or backbone-forming properties is preferably water-soluble.

The soap preparation—particularly in sheetlike or striplike configuration—has high flexibility, which is of great advantage particularly for application in the bodycare sector.

The soap preparation can be stored prior to use on a sheetlike support, from which it is removed directly prior to use. This sheetlike support may, for example, consist of board, cardboard, paperboard, a plastic such as polyethylene, polypropylene, polystyrene, polyester, ethylene-vinyl acetate copolymer or a composite material of these materials, for example a laminate of cardboard and polyester. The sheetlike support can contain grip aids (flaps, protruding sections etc.) and removal aids (perforations, cuts etc.) in order to facilitate removal of the soap preparation prior to use.

The soap preparation can also comprise at least one fragrance. The fragrances include: essential oils, natural fragrances and near-natural fragrances. Preference is given to water-soluble fragrances.

A soap preparation according to the invention can be prepared by the following general method: in a first step, the soap (which preferably also includes a foam former), at least one film- or backbone-forming polymer and the optionally desired further (solid and liquid) constituents of the soap preparation are mixed with water. The mixture is then stirred until a foamy mass of the desired consistency is formed. In this step, the air bubbles are thus introduced into the aqueous mixture of soap and film- or backbone-forming polymer. The nature of the stirring determines the number and the size of these air bubbles. The foamy mass is then preferably placed onto a weblike support, on which it can be transported into a drying tunnel. When producing a configuration of the soap preparation other than the sheetlike or striplike one, the foamy mass can, for example, also be poured into dies or deep-drawn blisters.

After drying, which preferably takes place in the drying tunnel and during which the water is removed from the foamy mass, the resulting, now solid soap preparation can be further processed by cutting length-ways, cutting crossways, punching, packaging, assembling etc. The residual water content in the solid soap preparation is preferably less than 10% by weight, particularly preferably less than 2% by weight. If desired, after the drying, an additional step of compression (compaction) of the soap preparation can also be carried out.

Sheetlike configurations of the soap preparation can also be obtained, for example, by punching or cross-cutting a material web into individual sections. These can in turn be packaged in sealed-edge bags, tubular bags, blister packs or vials.

The soap preparation can be used in toilet bowls, wash basins, washing machines, dishwashers, in body care, for cleansing the face, hair washing, hand washing, as WC cleaners, as kitchen cleaners, bath cleaners, window cleaners, multipurpose cleaners, stain removers, drain cleaners, sanitary cleaners, car window cleaners, floor cleaners, furniture care compositions, in the industrial sector, for the cleaning of shoes, as additive to window cleaning liquid containers in cars, in hotels, while traveling, in hospitals. Since the soap preparation is already predosed during the production into a single-use portion, the exact dose of a certain amount of soap can be ensured.

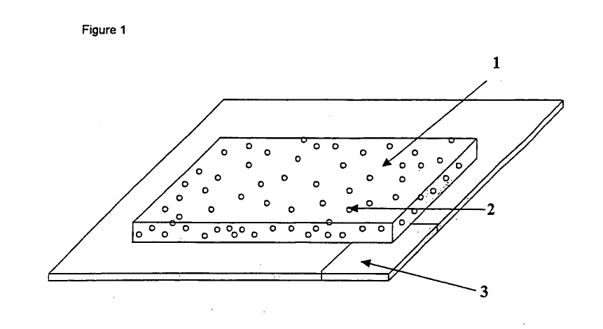

FIG. 1 shows a soap preparation in sheetlike configuration. Herein (1) is the mixture of soap and film- or backbone-forming polymer and (2) are the air bubbles in the soap preparation. The sheetlike support (3) has a cut which can serve as removal aid.

The preparation examples below serve to illustrate the invention.

### EXAMPLE 1

20 g of a polymer based on polyvinyl alcohol (as backbone-forming polymer; the polyvinyl alcohol is partially hydrolyzed, the degree of hydrolysis being between 80 and 95%, preferably between 85 and 92%, and a 4% strength aqueous solution of this polymer at 20° C. having a viscosity between 3 and 60 mPas, preferably between 5 and 40 mPas), 3.75 g of Lamepon S (potassium cocoyl hydrolyzed collagen), 1.25 g of Dehyton AB 30 (cocobetaine), 1.25 g of Dehyquart E-CA (N-(2-hydroxy-hexadecyl-1-)-N,N-dimethyl-N-2-hydroxyethylammonium chloride), 3.75 g of Texapon NSO (sodium laureth sulfate as foam former) and 10 g of Texapon N 70 (sodium laureth sulfate as foam former) are mixed with 20 g of water. This mixture is stirred at 25 rpm for 3 minutes. This gives a foamy mass which is applied to a traveling web made of polyamide in a spreading thickness of 1200 μm. The foamy mass is dried firstly for 20 min at 70° C. and then for 70 min at 90° C.

### EXAMPLE 2

16 g of the polyvinyl alcohol from example 1, 2 g of Lamepon S, 0.5 g of Dehyton AB 30, 0.5 g of Dehyquart E-CA, 4 g of Texapon NSO and 9 g of Texapon N 70 are mixed with 8 g of water. Stirring, application and drying are carried out as in example 1. This gives a solid soap preparation with a weight per unit area of 132 g/cm². (This corresponds to a density of about 0.11 g/cm³).

### EXAMPLE 3

A soap preparation is prepared as in example 2 except that the spreading thickness during spreading is 1600 μm. The resulting solid soap preparation has a weight per unit area of 257 g/cm². (This corresponds to a density of 0.16 g/cm³).

### EXAMPLE 4

A soap preparation is prepared as in example 1, where 20 g of the polyvinyl alcohol, 10 g of Lamepon S, 2.5 g of Dehyton AB 30, Dehyquart, 20 g of Texapon NSO, 45 g of Texapon N70 are mixed with 90 g of water. The foamy mass is spread in a spreading thickness (coating thickness) of 1800 μm. After drying, the soap preparation has a weight per unit area of 200 g/cm².

1. A soap preparation comprising a soap, at least one film- or backbone-forming polymer and air bubbles, characterized in that it is solid.

2. The soap preparation as claimed in claim 1, characterized in that it is elastic and plastic.

3. The soap preparation as claimed in claim 1, characterized in that the soap is a substance from the group of water-soluble sodium, potassium or ammonium salts of saturated or unsaturated higher fatty acids, of resin acids of colophony, of naphthenic acids, of surfactants or a mixture thereof.

4. The soap preparation as claimed in claim 1, characterized in that the film- or backbone-forming polymer is a natural and/or synthetic polymer.

5. The soap preparation as claimed in claim 1, characterized in that the film- or backbone-forming polymer is from the group of polyamides, polyacrylates, polyamino acids, polyvinyl acetate, polyvinyl alcohol, polyethylene glycols, polyvinylpyrrolidones, pullulan, alginic acid, starch, cellulose and cellulose derivatives or a mixture thereof.

6. The soap preparation as claimed in claim 1, characterized in that the soap is a foam former, preferably a surfactant or an interface-active substance.

7. The soap preparation as claimed in claim 1, characterized in that the air bubbles constitute 0.5 to 98% of the volume of the soap preparation.

8. The soap-preparation as claimed in claim 1 characterized in that it has a density below 1, preferably between 0.7 and 0.1 g/cm³.

9. The soap preparation as claimed in claim 1, characterized in that it is striplike.

10. The soap preparation as claimed in claim 1, characterized in that the expandability is less than 25%, preferably between 2 and 10%.

11. A method of producing a solid soap preparation which comprises a soap, at least one film- or backbone-forming polymer and air bubbles, comprising the steps:
a) mixing of soap and film- or backbone-forming polymer in water,
b) stirring this aqueous mixture in the presence of air giving a foamy mass,
c) coating a traveling web or filling hollow bodies or deep-drawn blisters with the foamy mass,
d) drying the coated traveling web or the filled hollow bodies or deep-drawn blister, during which the solid soap preparation is formed.

12. The method as claimed in claim 11, characterized in that an additional compression step can be carried out after the drying step.

13. The method as claimed in claim 11, characterized in that the resulting soap preparation is divided into individual doses.

14. The use of a solid soap preparation which comprises a soap, at least one film- or backbone-forming polymer and air bubbles for use in toilet bowls, wash basins, washing machines, dishwashers, in bodycare, for cleansing the face, hair washing, hand washing, as WC cleaners, as kitchen cleaners, bath cleaners, window cleaners, multipurpose cleaners, stain removers, drain cleaners, sanitary cleaners, car window cleaners, floor cleaners, furniture care compositions, in the industrial sector, for the cleaning of shoes, as additive to window cleaning liquid containers in cars, in hotels, while traveling, in hospitals.
